(19) United States
(12) Patent Application Publication
(10) Pub. No.: US 2004/0248111 A1
(43) Pub. Date: Dec. 9, 2004

(54) Screening method using solid supports modified with self-assembled monolayers

(76) Inventors: Gunther Metz, Heidelberg (DE); Holger Ottleben, Heidelberg (DE); Harald Rau, Heidelberg (DE); Nathalie Schellhaas, Ludwigshafen (DE); Renate Sekul, Ladenburg (DE); Dirk Vetter, Heidelberg (DE)

Correspondence Address: James F Haley Jr, Fish & Neave, 1251 Avenue of the Americas, New York, NY, 10020, US

(21) Appl. No.: 10/467,583

(22) PCT Filed: Feb. 5, 2002
(86) PCT No.: PCT/EP02/01185

(30) Foreign Application Priority Data
Feb. 7, 2001 (EP) 01102673.9

(51) Int. Cl. C12Q 1/68

(52) U.S. Cl. 435/6

(57) ABSTRACT

Disclosed is a method for the identification of active compounds interacting with a target molecule, comprising the steps of: (a) forming a binding matrix comprising at least two different ligands on a solid support by immobilising said ligands via a common intermediate molecule on the support; (b) contacting a target of interest with said binding matrix; (c) parallely determining a binding value of the ligand/target interaction for each type of ligand comprised in the binding matrix; (d) selecting those ligands the binding value of which in an immobilised state with the target exceeds a predetermined threshold; (e) replacing the common intermediate molecule by a replacing fragment in order to form new ligands of increased molecular weight, with the replacing fragment being coupled to the ligand(s) of step (d) at the common binding position between the ligand and the intermediate molecule; (f) determining the affinity of the ligands formed in step (e) towards the target.

The present invention relates to a method for the identification of active compounds which are capable of interacting with a target molecule.

Drug discovery processes usually involve the task of finding a compound which, due to its specific chemical structure, interacts with a target molecule showing corresponding structural features in order to increase or decrease the activity of the target. Usually, targets are of natural origin, such as proteins. Once such a compound has been identified, medicinal chemistry helps to modify its structure in a way which, eventually, yields an active compound for a pharmaceutical composition. However, since the identification of suitable interacting partners for a target is relatively time consuming, methods are being investigated in order to accelerate the retrieval of molecules with active structures.

One approach in this respect is the screening, particular the high throughput screening (“HTS”), which is based on the principle of increasing the probability for the successful identification of suitable compounds by testing of a large number of molecules (also referred to as ligands). In order to be able to test hundreds or thousands of compounds on a short time scale, screening assays used in HTS are usually based on microplate systems and robotic liquid handling technology. Using such a HTS method, a large variety of potential drug candidates can be tested for their capability of interacting with a target. However, the vast palette of available reagents strongly increases the size of theoretically accessible libraries of chemical compounds to be tested, before a promising candidate can be found among them. Moreover, the task of providing suitable compound libraries for screening methods has become more difficult as a result of the human genome project due to which more targets are available than can be studied by X-ray crystallography, nuclear magnetic resonance or other high resolution biophysical techniques. Therefore, a very large number of targets, for which in most cases no functional data are available, need to be studied without suitable information regarding preferred structural features of the compounds are to be tested.

Thus, another approach concentrates on the purposive selection of compounds subjected to screening. This strategy equally starts from a large group (or library) of accessible compounds. Generally, the compounds included into a library for this purpose are either commercially available or easy to synthesise, for example via methods of combinatorial chemistry.

Subsequently, large libraries are reduced to smaller subsets which requires a selection of a set of compounds most representative of the entire library. This process of compound selection is called “library design”, and is usually accomplished randomly, guided by medicinal chemistry or computer-aided. “Representative” in this context means that, taking into account the criteria applied for compound selection, the members of this subset are as closely related as possible to the remaining compounds in the original library. On the other hand, structural diversity should be as high as possible within these subsets. Suitable selection criteria are one- or higher dimensional descriptors, such as molecular weight, number of bonds which can rotate freely, hydrophobicity, etc. or a combination of these. The obvious advantage of selecting a subset of compounds, that best represents the full range of the chemical diversity present in the larger population, is to avoid the time and expense of synthesising and screening redundant compounds.

Finally, a further approach aims at the identification of suitable structures among compounds which, in spite of their low molecular weight and their limited complexity, show already a certain affinity towards a target molecule. Via purposive combination of such low-affinity ligands, active compounds are synthesised. This approach has the advantage that the number of potential starting compounds is usually limited compared to cases where highly complex ligands, consisting of a large number of potentially active fragments, are tested for their affinity. However, it brings about the problem that additional information is required in order to allow the effective combination of the low-affinity ligands or fragments retrieved. Thus, if the size of a low affinity ligand is increased by chemically coupling to another compound, the reaction may easily block the active site of the ligand, thus giving rise to a decrease rather than an increase in affinity.

For solid phase screening, either the predominantly macromolecular target or the candidate target binding molecule, i.e. the ligand can be immobilised. While the first alternative is already in use in order to detect high affinity binding partners, the second alternative of immobilising the ligand is considered to be unsuitable for screening because of the putative steric hindrance of the interaction target (see Gordon E. M. and Kerwin J. F., Combinatorial Chemistry and Molecular Diversity in Drug Discovery, Wiley-Liss 1998, p. 424-425).

The object of the invention is thus the provision of a method which combines the advantages of the above approaches to increase the probability of retrieving an active compound.

Prerequisite for carrying out the present invention is the immobilisation of those chemical compounds which are to be tested for interactions with a certain target molecule. If a plurality of (different) chemical compounds (or ligands) is immobilised, the method may be referred to as a solid phase screening method. It is, however, disadvantageous if the compounds are directly attached to the solid phase surface, since the solid support might affect interactions with the target. For this reason, the ligands are immobilised via intermediate molecules and/or ligand-tags, to which they are covalently bound. After testing the immobilised ligands for their potential to interact with the target in a first screening step, the most promising candidates are further modified to yield ligands of increased molecular weight, which may, theoretically, show an increased affinity to the target.

Surprisingly, it was found that specifically the replacement of the intermediate molecule by another compound (replacing fragment) leads to a more active ligand at a higher probability than any other synthesis step yielding molecular structures of higher complexity. In other words, only if the compound to be newly introduced is bound to the exact position previously taken by the intermediate molecule, a retention or even an increase of activity of the new combination, compared to the original ligand, is highly probable. Because of the directed presentation of the ligands caused by the intermediate molecules comprised in the chemical surface, the replacement of said intermediate molecules permits the same interaction between the original part of the ligand after introduction of a replacing structure and the target.

In a subsequent step, the resulting ligands of increased molecular weight are again tested for their ability to interact with the target molecule to proceed towards an active compound which shows sufficient affinity to be used in a pharmaceutical composition. The test can be carried out, e.g. as a solid phase screening process or in solution with the ligand in a free, non-immobilised form. Such values obtained e.g. in solution according to conventional methods from the equilibrium between free ligands and targets on the one hand and ligand—target complexes on the other hand are characteristic indicators for the in vivo effectiveness of a chosen ligand. If, on the other hand, the combinations of the replacing fragment(s) and the original ligand are coupled to a solid support for screening, the method of the invention can be reiterated until a compound of sufficient activity has been found.

As a consequence, the method according to the invention comprises the steps of:

(a) forming a binding matrix comprising at least two different ligands on a solid support by immobilising said ligands via a common intermediate molecule on the support;

(b) contacting a target of interest with said binding matrix;

(c) parallely determining a binding value of the ligand/target interaction for each type of ligand comprised in the binding matrix

(d) selecting those ligands the binding value of which in an immobilised state towards the target exceeds a predetermined threshold;

(e) replacing the common intermediate molecule by replacing fragments in order to form new ligands of increased molecular weight, with the replacing fragments being coupled to the ligand(s) of step (d) at the common binding position between the ligand and the intermediate molecule

(f) determining the affinity of the ligands formed in step (e) towards the target.

In case the replacing fragments used in step (e) to modify the original ligands comprise a common structure which may serve as an intermediate molecule to immobilise the new ligands to a solid support, steps (a) to (e) may be reiterated until a ligand with a sufficiently high affinity towards the target is formed. Also, ligands which have already undergone the affinity determination of step (f) may be reintroduced into the method of the present invention, if desired. Suitably, such ligands can be used among starting compounds in step (a) or among the ligands selected in step (d).

The support used to immobilise the ligands comprises a solid substrate such as a metal or an inorganic non-metal, metal- or non-metal oxides such as alumina, silicates or glasses, etc., an organic matrix, such as cellulose or membranes (e.g. made from polypropylene, polystyrene, or suitable mixtures of the above materials. Preferably, the support is formed by a metal, most preferably a noble metal (silver, palladium, platinum; especially gold) or a substrate the surface of which is at least partly covered with a layer of such a metal. The material used depends on the detection method. If reflection-optical methods, such as surface plasmon resonance (SPR) are used, the preferred substrates are glass or a light transmitting polymer coated with a thin gold film.

The ligands are immobilised on such a support to form a binding matrix, and in the context of the present invention, the term “binding matrix” generally refers to a surface comprising a plurality of different immobilised ligands. Normally, a plurality of different types of ligands, such as at least 1000, or even at least 9000, is immobilized in step (a). Preferably, the number of different types of ligands is at least 1536, 3072, 4608 or even 9216.

Preferably, the immobilised ligands are arranged in a two-dimensional array formate, i.e. on a microarray comprising discrete fields the spatial location of which can be easily identified and addressed. Each location of the array carries groups of one type of ligand from a known source and with a known structure. Suitable microarrays for the purpose of the present invention include, e.g., a two-dimensional planar solid support with a plurality of position-addressable reaction areas for the immobilisation of samples of small size, preferably in a regular pattern, of about less than 2.5 mm, preferably less than 1 mm, more preferably 0.5 mm in diameter, for screening purposes. As regards the number of reaction areas, conventional microtiterplate formates can be used, such as those of the 96-well or 384-well type. However, in terms of an acceleration of the screening process, the number of reaction areas preferably reaches at least 1536, more preferably at least 3072 or at least 4608 and particularly preferred are 9216.

If a microarray is used as a solid support, the number of different compounds in the initial library of candidate target binding molecules preferably corresponds to the number of reaction areas in the array. For the method of the present invention, libraries comprising at least about 1536, particularly at least 3072 or at least 4608, more particularly at least 9216 different compounds are preferred.

In order to form a suitable binding matrix, the intermediate molecules should be part of a homogeneous layer on the solid support so as to avoid detectable interactions of the target with these molecules. This aim can best be achieved if the same intermediate molecule is common to all the immobilized ligands. In this case, differences in the binding value determined in step (c) can be attributed to the differences in the ligand structure, and significant contributions by the interaction between the target and the intermediate structure can be excluded. It should thus be understood that the term “common intermediate molecule” as used above relates to the potential of these molecules to interact with a given target. Accordingly, minor variations between the chemical structures of the intermediate molecules, which cannot be expected to significantly influence their overall steric or electronic structure are still included within the scope of the invention. Nevertheless, particularly preferred is the case where all the ligands tested in the first screening step are immobilized on the solid support via intermediate molecules which are chemically fully identical.

Possible steric effects that might have a negative influence on the determination of the binding value, such as steric hindrance between bound targets or between targets and ligands as well as spurious signals resulting from unspecific binding between targets and ligand clusters are preferably avoided by using a special surface chemistry. When this strategy is applied, “dilution components”, i.e. structures that do not act as ligands, are preferably comprised in the binding matrix. Such dilution components present structures within the binding matrix, which, due to their low steric or electronic complexity, cannot be expected to bind to the target of interest. Rather, these components serve exclusively to spatially separate the ligands. Therefore, the dilution component should have a high adsorption resistance towards the target, e.g. a protein. If dilution components are used, the binding matrix presented to the target is well structured, with a controlled density of ligands helping to avoid agglomerations of ligands and ligand-ligand interactions. Moreover, the ordered structure of the molecules forming this binding matrix strongly reduces background signals arising from unspecific binding between the target and the support or the target and the ligands. Preferably, the same type of dilution components should be used throughout the binding matrix in order to fully exclude any influence of these compounds on the determination of the binding value of the ligands. In order to ensure homogeneous mixing of the dilution components and the intermediate molecules carrying the ligands on the support, it is further preferred to use dilution components which are structurally as similar as possible to the intermediate molecules.

Intermediate molecules and dilution components for the purpose of the present invention are preferably substantially linear and possess a structure which allows them, due to interaction with the adjacent molecules, to align themselves regularly, on the surface of the support. Preferred are self-assembling monolayers (SAM). They are formed, e.g. by hydrocarbon chains, which are substantially linear, optionally interrupted by heteroatoms and/or amide and/or ester bonds. Normally, the chains comprise 2 to 50, preferably 5 to 30 C-atoms. Such a SAM is very resistant to unspecific target-adsorption which strongly reduces the background. In order to allow their immobilisation, the intermediate molecules and the dilution components provide a functional group, preferably at one terminal of the linear structure the chemical nature of which depends on the material used as a support. Thus, sulfur containing compounds, such as thiols, are preferably applied to gold surfaces.

For their covalent coupling to the ligand, the intermediate molecules are further required to provide a second functional group, which may react with a corresponding structure of the ligand. Non-limiting examples for supplementary chemical functionalities are the combinations of a carboxylic group and an amine, a carboxylic group and an alcohol, a sulfonyl acid and an amine. It is well known in the art to use such functional groups directly or in activated form (eg. an acid halide, an anhydride, the reaction product of the carboxylic acid with a carbodiimide or an ester with N-hydroxysuccinimide instead of the carboxylic acid group).

Suitable structures to be used as intermediate structures and dilution components for the purpose of the present invention are, e.g., the anchor structures disclosed in WO 00/73796 and DE 100 27 397.1, and those are preferred for the purpose of the present invention which carry a thiol functionality to interact with the solid support. Suitable structural elements that support SAM formation and, at the same time, allow the adjustment of suitable distances between the support and the ligand, are described in DE 199 24 606.8 or WO 00/73796. The above documents also provide a detailed description of methods for the synthesis of such anchors and of suitable binding matrices containing them together with ligands attached to them.

The ligand may be bound to the anchor structure prior to its immobilisation on the support. In this case, complete ligand-anchor-conjugates (LAC) are contacted with and bound to the support as disclosed in WO 00/73796.

However, for the present method, the strategy disclosed in DE 100 27 397.1, where the anchor molecules are immobilised on the support in an activated form and are subsequently bound with the ligand, has proven to be particularly advantageous. In this latter approach, anchor structures are synthesized so as to carry a reactive “head group”, i.e. a group which allows a selective and preferably quantitative reaction of the thus activated anchor with the ligand. It should be understood that this head group is at a terminal of the anchor structure facing away from the support on which the anchor is immobilized. Depending on the chemical nature of the head group, this strategy may require a chemical modification of the ligand so as to carry a specific functionality which is able to react with the head group of the activated anchor. Once the activated anchors are immobilised on the solid support, they can be reacted with the ligand/modified ligand in a separate step to provide the final binding matrix. The ligand/modified ligand is preferably added in an amount at least as high as that of the anchor molecules present on the surface to reach complete conversion of the “head groups”. An advantage of this method is that the ligand concentration is solely determined by the concentration of anchor molecules and not by the concentration of ligands in the added solution. This is of particular advantage if many ligands that are e.g. obtained by combinatorial synthesis and that are present in imprecise concentration, have to be analysed in parallel. Therefore, the reproducibility and the comparability of different measurements can be improved. Mercaptophilic head groups as exemplified in DE 100 27 397.1 which covalently bind the ligand are preferred for this purpose. Among them, the method of providing a binding matrix by reacting a thiol-containing ligand with immobilised anchors carrying a maleimide as a head group has been proven particularly advantageous. In this case, a thiol functionality is contained in or bound to the ligands to be screened. Once the surface of the support is covered with the anchor structures, the thiol—functionalised ligands are reacted with the mercaptophilic head group to provide ligand anchor conjugates immobilised on the support.

Thus, in a first preferred embodiment, intermediate molecules of the present invention have the following general structure

HS—R-M (1)

for the definition and synthesis of which, including particularly preferred embodiments, it is referred to DE 100 27 397.1 according to which R is a linear or branched, optionally substituted, saturated or unsaturated hydrocarbon chain which may comprise heteroatoms, aromatics and heterocyclic compounds. It comprises 5-2000 atoms, including heteroatoms. In a preferred embodiment, R in formula (1) comprises one or both of the structural subunits R^{a} and R^{b}, with R^{a} being positioned adjacent to the thiol functionality.

R^{a} is a bivalent moiety, which preferably allows the formation of a SAM and for this purpose it should be largely hydrophobic. It comprises a branched or linear hydrocarbon chain of 5 to 50 carbon atoms which may be completely saturated or partly unsaturated and which may be interrupted by aromatics, heterocyclic compounds or heteroatoms, a completely saturated hydrocarbon chain without heteroatoms being preferred. In a preferred form, it has the general formula —(CH₂)ₙ—, wherein n is an integer from 5 to 50, preferably from 5 to 25, particularly preferably from 5 to 18 and most preferably from 8 to 12.

R^{b}, which is equally bivalent, represents in a first preferred embodiment an oligoether of the general formula —(OAlk)_{y}—, wherein y is an integer and Alk is an alkylene group. A structure wherein y ranges between 1 and 100, preferably between 1 and 20, and most preferably between 2 and 10, is preferred. The Alk group preferably exhibits 1-20, more preferably 2-10 and particularly preferably 2-5 carbon atoms. —(OC₂H₄)_{y}— is most preferred.

In a second preferred embodiment, R^{b} is an oligoamide which is formed by dicarboxylic acids and diamines and/or amino carboxylic acids, wherein the amines independently of each other exhibit from 1 to 20, particularly preferably from 1 to 10 carbon atoms and may also be interrupted by further heteroatoms, in particular oxygen atoms. The carboxylic acid monomers, independently of each other, preferably have from 1 to 20, more preferably from 1 to 10 carbon atoms and may also be interrupted by further heteroatoms, in particular oxygen atoms.

Further preferred are anchor structures wherein either R^{a} alone, or R^{a} and R^{b} together, link HS and M in the above formula (1).

Particularly preferred are groups R of the general formula:

—(CH₂)ₐ-Q¹-(CH₂)_{b}-{[Q²-(CH₂)_{c}—[O—(CH₂)_{d}]ₑ—O—(CH₂)_{f}]_{g}—[Q³-(CH₂)_{c′}—[O—(CH₂)_{d′}]_{e′}—O—(CH₂)_{f′}]ₕ}ᵢ-Q⁴-(CH₂)ⱼ-Q⁵-(CH₂)ₖ— (2)

wherein the variables, independently of each other, are defined as follows and numerical ranges are to comprise their respective limiting values as well as all integers in-between:

Q¹, Q⁵ represent —NH—C(O)—, —C(O)—NH— or a bond;

Q², Q³, Q⁴ represent —NH—C(O)— or —C(O)—NH—;

a is from 5 to 20, preferably 8 to 12, particularly preferably 10;

b is from 0 to 5, preferably 0 if Q¹ is a bond and from 1 to 10, preferably 2 to 7, particularly preferably 3 to 5 in all other cases;

c, c′ are from 1 to 5, preferably 1 to 3, particularly preferably 1;

d, d′ are from 1 to 5, preferably 1 to 3, particularly preferably 2;

e, e′ are from 1 to 5, preferably 1 to 3, particularly preferably 2;

f, f′ are from 1 to 5, preferably 1 to 3, particularly preferably 1;

g, h are from 0 to 3, provided that g+h≧1, preferably g+h=2;

i is from 1 to 3, preferably 1 to 2, particularly preferably 1;

j is from 0 to 5, preferably 1 to 3, particularly preferably 2; and

k is from 0 to 5.

Mercaptophilic head groups M are, e.g., iodine and bromine acetamides, pyridyldithio compounds, Michael acceptors in general, acrylic acid derivatives such as the esters, amides, lactones or lactames thereof, methylene-gem-difluorocyclopropanes, α,β-unsaturated aldehydes and ketones as well as α,β-unsaturated sulfones and sulfonamides.

Preferred head groups M are those of the general formula

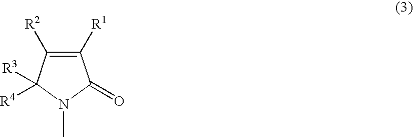

wherein

R¹ and R², independently of each other, represent hydrogen or C₁-C₅ alkyl, preferably methyl, ethyl or n-propyl,

R³ and R⁴, independently of each other, represent hydrogen or C₁-C₅ alkyl, preferably methyl, ethyl or n-propyl, or R³ and R⁴ together are ═O and

the binding to the other anchor is effected via the nitrogen atom.

Preferably, R³ and R⁴ together are ═O, most preferably the head group is a maleimidyl group.

For the purpose of binding the ligand to an anchor molecule, in particular an activated anchor molecule (e.g. of formula (1)) already immobilised on the support used for screening, it is preferred that the ligands are supplied with a specific structure (“ligand-tag”). In this case, both the anchor and the ligand-tag, once combined with the ligand, may be considered to form the intermediate molecules of the present invention.

The structure of such a ligand-tag may be depicted by the formula:

Z-A-Y, (4)

wherein

A is a chemical bond or a hydrocarbon chain of 2 to 50, preferably 5 to 30 C-atoms, optionally interrupted by heteroatoms, amide or ester bonds,

Y is a functional group to react with the ligand, and

Z is a functional group which is able to react with the head group of (the) a corresponding anchor molecule, preferably a thiol, carboxyl or amino group. Particularly preferred is a thiol, capable of reacting with a mercaptophilic head group of the anchor molecules as described above.

Preferably, A is unbranched to minimise unspecific interactions between the “ligand tag” and the target. Heteroatoms suitable for A comprise O, N, S, Si, P, B.

Preferred are groups A of the general formula:

—(CH₂)ₗ-Q⁶-[(CH₂)ₘ-Q⁷]ₙ-(CH₂)ₒ-Q⁸-[(CH₂)_{m′}-Q⁹]_{n′}-(CH₂)_{o′}-Q¹⁰-(CH₂)_{p′}— (5)

wherein the variables, independently of each other, are defined as follows and numerical ranges are to comprise their respective limiting values as well as all integers in-between:

Q⁶ to Q¹⁰ represent independently —NH—C(O)—, —C(O)—NH—, —NH—C(O)—O—, —O—C(O)—HN—, —C(O)—O—, —O—C(O)—, a heteroatom or a bond;

l, p, p′ are independently integers from 0 to 5, preferably 0 to 3;

m, m′, o, o′ are independently integers from 1 to 5, preferably 1 to 3, particularly preferably 2;

n,n′ are independently integers from 0 to 20, preferably 2 to 15 and particular preferably 3 to 10, with the proviso that at least one of n and n′ is not 0.

More preferably, A comprises at least 1 amide bond and at least 4 heteroatoms. Particularly preferred is an A comprising two amide bonds and four oxygen atoms.

Examples for Y are primary and secondary amino groups, carboxylic acid groups, hydroxyl groups, hydroxylamino groups, ester, aldehyde and other carbonyl moieties. Preferably, Y is —NH₂, —NHR⁵, —NR⁵OH, —C(O)H, —C(O)OR⁵, or —C(O)OH, wherein R⁵ is a C₁-C₆ alkyl group such as methyl, ethyl, n-propyl, i-propyl etc. Most preferably, Y is a primary amino group. Examples for supplementary chemical functionalities on the ligand to which Y may be reacted to form a covalent bond are the combinations of a carboxylic group and an amine, a carboxylic group or an ester and an alcohol, a sulfonyl acid and an amine. It is well known in the art to use such functional groups directly or in activated form (eg. an acid halide, an anhydride, the reaction product of the carboxylic acid with a carbodiimide or an ester with N-hydroxysuccinimide instead of the carboxylic acid group). However, many chemical reactions can be used to bind the ligand-tag Z-A-Y to the ligand and to the anchor molecule so the provided examples are not limiting. One skilled in the art can extend the list of examples and knows the chemical reactions like addition reactions, substitution reaction and condensation reactions leading to the desired chemical bond with the ligand.

The selection of the optimum ligand-tag for the inventive method depends on the ability of the ligand-tag to (a) minimise unspecific binding of the target to the ligand-tag, (b) present the target in a suitable distance from the SAM to the target to avoid steric repulsion between the SAM and the target and (c) provide a high mobility of the ligand for optimum binding capability. The selection of the ligand-tag also depends on the size and chemical nature of the target. Such ligand-tags, if used, are either directly attached to the ligand during its synthesis or immediately prior to its coupling with the anchor molecule. In a preferred embodiment, each immobilised ligand possesses the same ligand-tag.

Particularly suitable binding matrices to be used in step (a) of the method according to the invention are obtained if the dilution components and the ligand carrying intermediate molecules are present on the support in a ratio ranging from 1:2 to 1:10000, preferably from 1:10 to 1:1000 or 1:10 to 1:100. Homogeneously functionalized surfaces are best provided by bringing a well mixed solution of both intermediate molecules and dilution components in contact with the support.

The total length of the dilution components should be slightly shorter than that of the intermediate molecule. Otherwise, the anchor molecule and the dilution component should have a large structural similarity in order to ensure homogeneous blending on the solid phase surface and to allow the formation of well structured SAMs. These criteria are fulfiled, e.g. in the case where activated anchor structures together with the ligand-tags form the intermediate structure, and dilution components are used which structurally resemble the anchor structure alone. Exemplary dilution components for this approach have the general formula

HS—R—X (6)

the variables of which are equally defined in DE 100 27 397.1, and they are preferably used for the purpose of the present invention. Thus, while R is independently defined as for the anchor structure above, X is a non-mercaptophilic head group, preferably derived from a small molecule with a molecular weight of less than 50, 40 or even 30 g/mol. Often, C₁-C₄ alkoxy or acylamide groups are used and methoxy groups as well as acetamide groups are particularly preferred. Here, the dilution components and the anchor molecules are preferably used in a ratio ranging from 1:2 to 1:10000, more preferably from 1:10 to 1:1000 and particularly preferable from 1:10 to 1:100. Again, homogeneously functionalised surfaces are best provided by bringing a well mixed solution of both anchors or intermediate structures and dilution components in contact with the support, and it is referred to DE 100 27 397.1 with regard to specific techniques. After this step, the ligands can be bound to the anchor structures. Alternatively, such preferred dilution components can also be used in cases where complete ligand anchor conjugates as described e.g. in WO 00/73796 are used to form the binding matrix. Here, mixed solutions comprising the dilution components together with ligand anchor conjugates are contacted with the support.

Suitable intermediate molecules to be further modified to carry ligands, conjugates of intermediate molecules or ligand-tags and ligands as well as dilution components are preferably provided by solid phase synthesis, followed by cleaving the desired product from the solid substrate used during its synthesis and contacting it with the solid support used for screening.

Suitable ligands for the purpose of the present invention are, in principle, all compounds which can be expected to interact with any target molecule of interest. Among the vast number of compounds included in this group, a selection can be made using, eg. criteria for library design known in the art, like diversity, drug- or lead-likeness (for the latter criterion, cf. Teague et al., Angew. Chem. Int. Ed. 1999, 38: 3743-48), commercial or synthetic availability.

For the selection of ligands according to their “drug-likeness”, the ADMET rules may be applied, which associate drug-like properties with the criteria of Absorption, Distribution, Metabolism, Excretion and Toxicology. These are most commonly defined using the “rule of 5” based on properties of known drugs (Lipinski, C. A. et al., Adv. Drug Deliv. Rev. 1997, 23: 3-25). According to the Lipinsky rule, a given ligand does not form a suitable basis for the development of a drug if the ligand as such or a compound incorporating it fulfills two or more of the following criteria:

(1) molecular weight >500

(2) Number of hydrogen-bond acceptors >10

(3) Number of hydrogen bond donors >5

(4) Calculated logP >5 (if ClogP is used) or >4.15 (if MlogP is used)

Compounds failing this scheme are assumed not to show an absorption or permeation suitable for a drug.

For application of the Lipinsky rule, all oxygen and nitrogen atoms are considered as hydrogen-bond acceptors and N—H or O—H groups are defined as hydrogen bond donors. LogP refers to the octanol/water partition coefficient of a given compound and is used to estimate ist lipophilic properties. Different approaches for the calculation of logP have been developped, such as ClogP (Dailight Information Systems) and MlogP (Moriguchi, I. et al., Chem. Pharm. Bull. 1992, 40, 127-130).

Following the lead-like classification coined by Teague et al, the molecules initally immobilized in step (a) should have an number-average molecular weight of less than 400, preferably <380, more preferably <370 and most preferably <350 g/mol mol wherein an individual ligand can have a significantly higher molecular weight, preferable less then 800, more preferred less then 700 g/mol. Also preferred are libraries wherein the ligands share a small common core size. In particular, ligands obtained by forming binary combinations from two sets of reactants, which are directly connected as building blocks e.g. in a step of combinatorial synthesis are preferred. The reactants forming the building blocks then have typical molecular weights ranging from 50 or 75 to 250, preferably from 100 to 150 or 200, particularly preferred from 150 to 200 g/mol.

Computational methods for ligand selection (Pearlman and Smith, 1998; Jamois et al. 2000) help to cope with the large number of potential compounds which can be synthesised by combinatorial chemistry and which exceeds screening capacities even in the context of ultra high-throughput technologies. In order to describe the complex properties of compound collections such as molecular diversity or structural bias towards a pharmacophoric motif, several molecular encoding schemes have been developed that can be used for computerised storage and processing. Molecular descriptors range from simple whole molecule properties (molecular weight, clogP, polarisability) to 2D descriptors representing atom connectivity's (structural keys, fingerprints) and to methods for capturing 3D information (pharmacophore fingerprints). Conceptually, molecules are distributed in a high-dimensional so-called diversity space which is defined by a set of descriptors. Common mathematical methods for compound selection are either based on intermolecular distance together with clustering algorithms. Alternatively, cell-based partitioning methods with prior reduction of the dimensionality are applied (Gorse D. and Lahana R., Current Opinion in Chemical Biology 2000, 4: 287-294; Van Drie J. H. and Lajiness M. S., Drug Discovery Today 1998, 3: 274-283).

Preferred ligands to be used in the context of the present invention comprise a structure of the following general formula:

L¹-L², (7)

wherein L¹ and L² represent building blocks which are independently formed by an amine, alcohol, carboxylic acid or an amino acid, chosen such that the reactants yielding L¹ and L² have supplementary chemical functionalities which allow the direct formation of a chemical bond. For the method of the present invention, preferably the ligands are not formed from two natural occurring amino acids connected by the condensation reaction of the alpha amino group of one amino acid with the alpha carboxyl group of the second amino acid in the same ligand. Ligands based on those dipeptides can be sensitive to enzymatic degradation during the screening method of the present invention. Drugs developed on the base of those dipeptides are expected to be sensitive to enzymatic degradation resulting in short in vivo half live times.

The ligand is synthesised from two reactants Lᵣ¹ and Lᵣ² (preferably belonging to two different reactant libraries) which yield the corresponding building blocks L¹ and L², respectively. They contain at least one functional group suitable for the synthesis of the desired combinatorial library of ligands and Lᵣ² contains at least one additional functionality or functional group suitable to covalently bind the ligand to the intermediate molecule. The functional groups of Lᵣ¹ and Lᵣ² required for their combination can be independently an amine, an alcohol, a thiol, a carboxylic or a sulfonic group, chosen such that Lᵣ¹ and Lᵣ² have supplementary chemical functionality which allow the direct formation of a chemical bond. Non-limiting examples for supplementary chemical functionalities are the combinations of a carboxylic group and an amine, a carboxylic group and an alcohol, a sulfonyl acid and an amine. It is well known in the art to use such functional groups directly or in activated form (eg. an add halide, an anhydride, the reaction product of the carboxylic acid with a carbodiimide or an ester with N-hydroxysuccinimide instead of the carboxylic acid group). Moreover, the reactants Lᵣ¹ and Lᵣ² may comprise protective groups in order to avoid reactions of further functional groups which are to serve for the immobilisation of the ligand or potential interaction with the target. During synthesis or at the end of the synthesis of the ligand, the protective groups can be removed. Protective groups for organic chemical synthesis are known by one with ordinary skills in the art including the reagents and conditions for their introduction and for their removal.

The functional group of L² required for the attachment of the ligand to the intermediate molecule can be an amino, a hydroxyl or a thiol group, a carboxylic acid or a sulfonic acid residue or any other functionality of a chemical component capable of forming a covalent bond to a corresponding supplementary functionality.

Beside the required functional groups for the synthesis of the ligand and their attachment to the intermediate molecule, the reactants Lᵣ¹ and Lᵣ² can contain additional functional groups which may be introduced in a protected form to avoid side reactions during the synthesis of the ligand. Such functional groups represent potential sites for the interaction with the target. Non-exclusive examples for funcional groups are —OH, —SH, —S—C1-4-alkyl, —Cl, —F, —Br, CF3, —CN, —CHO, COOH, —COO—C1-4-alkyl, —C1-4-alkyl, —C1-4-alkyloxy, —NO2, —NH2, —NH—C1-4-alkyl, —CONH2, —COHN—C1-4-alkyl, —CON—(C1-4-alkyl)2, —NHCO—C1-4-alkyl, aryl, heteroaryl.

Although the inventive screening method can be generally used with a wide range of different targets, the screening method is preferably used for the screening of enzymes and particularly useful for the screening of proteases. Proteases catalyse the cleavage of peptide bonds. Ligands synthesised from an amino acid and a carboxylic acid or sulphonic acid own certain molecular elements common to naturally occurring peptides. Thus, it can be expected that they are able to bind specifically to the active site of proteases and that they are not cleavable at all or not with the same reaction rate by proteases as are natural occurring peptides. Ligands suitable for the inventive screening method should not be cleavable during the screening process by the target to avoid misleading results. Thus in a more preferred embodiment, the Lᵣ¹ is a reactant containing a carboxylic acid group or a sulfonic acid group function. Lᵣ² is an amino acid or amino acid with protective groups where appropriate and the immobilisation is accomplished by a carboxylic functionality of the amino acid. By varying the reactant Lᵣ¹ (“cap”) of these so-called “capped amino acids” the diversity space of the combinatorial library can be increased.

In order to connect the ligand tag, if used, and the ligand, the functional group Y of formula (4) is reacted with a suitable functional group of Lᵣ², preferably the one which is described above as serving for the immobilisation of the ligand on the solid support used for screening, e.g. an amino group, a hydroxyl group, a thiol, a carboxylic acid, a sulfonic acid. In preferred cases, where Lᵣ² is an amino acid, its carboxylic group can be used for this purpose. In this case, Y preferably represents an amine to form an amide bond with Lᵣ²

Following their immobilisation, the ligands are brought into contact in step (b) with a solution of the target of interest. Suitable targets for which the method of the present invention is particularly useful are macromolecules, in particular biomacromolecules. Preferred targets are proteins, DNA, RNA, oligonucleotides, prosthetic groups, vitamins, lipids, oligo- or polysaccharides, but also synthetic molecules, such as fusion proteins or synthetic primers. Particularly preferred are proteins, such as a protease.

Ligand/target interactions can be detected by any system comprising a support on which chemical compounds are immobilized, using, e.g., electrochemical, radiochemical, mass-sensitive or optical methods, such as fluoresence or luminesence measurements. Of course, methods allowing the parallel detection by means of a suitable imaging system, such as a CCD camera, are preferably applied. Moreover, direct binding assays are advantageous.

The choice of the mode of detection is an important element in surface-based techniques for the screening of binding interactions. Suitable labelling methods for the detection of target-ligand interactions on a solid surface are radio-immunoassays and optical methods, as for example fluorescence or luminescence measurements (especially enzyme assays). In a preferred embodiment, the so-called ELISA technique (enzyme-linked immunosorbent assay), an immunoassay on solid phase, is used. Here, the solid support is used solely for the immobilisation of one interaction partner.

However, labels used in these approaches may have the disadvantage of influencing specific binding interactions. Besides, labelling requires extra synthesis and isolation steps. Considering the many new proteins that are or will be delivered from the isolation or expression of human genes, the possibility of label-free detection of interactions with small amounts of protein sample is desirable (see Haake et al. (2000), J. Anal. Chem. 366, 576-585). Suitable methods for the label-free detection of target-ligand interactions are reflection optical techniques. Reflection-optical methods comprise surface plasmon resonance (SPR) and reflective interference spectroscopy (RlfS). In these methods, the solid support is an integral part of the sensor system.

Surface plasmon resonance (SPR) detects changes in refractive index that occur at the transducer surface during the binding event under investigation. In this method, an optical support (preferably a prism) is covered with a thin metal film and the change in intensity of the intern at the prism reflected light that occurs upon ligand-target binding is measured as function of the wavelength or as function of the adjusted angle. The SPR method has proven to be very useful in various fields and is now an established technique. Reflective interference spectroscopy (RlfS) is capable of using the partial reflection of light at interfaces for detecting changes in layer thickness. The attachment of biomolecules to binding partners (ligands) causes a shift in the intensity profile as a function of the wavelength. The shift of the detected curves is proportional to the change in layer thickness. Another label-free method are biosensors based on quartz micro balances. The bonds between targets and ligands are measured by means of the weight increase affecting the frequency of oscillating quartz crystals (Ebara and Okahata, JACS 2000, 116: 11209-12). However, in a preferred embodiment, the detection technique for ligand-target interaction during the method of the present invention is surface plasmon resonance (SPR).

After screening the immobilised ligands with regard to their potential to bind to the target, ligands of interest are selected in step (d) by defining certain thresholds of the binding value obtained in the screening process. Preferably, the binding value, which represents a relative value for the affinity between the ligands and the target, is obtained by presenting to the target molecules a binding matrix which comprises a plurality of ligands of each of the different types and detecting binding events with the above methods. The more target molecules bind with the representatives of one type of ligand, the higher is the binding value of this type of ligand. In the claimed method, hits are preferably selected by ranking the molecules pursuant to their binding values, and the threshold of step (d) of the method according to the invention may be deliberately chosen as to include a certain partition of the screened ligands in the replacement reaction of the following step (e).

In the next step (e), the common intermediate molecule of the ligands selected in step (d) is replaced. In this context, it must be understood that this replacement needs not to be carried out at the actual ligands coming from the screening process. Rather, only the structure of the new compounds combined from the original ligands and replacing fragments must correspond to a structure obtained if the concerned common intermediate molecule was replaced. The compounds as such can of course be newly synthesized, which is in fact the preferred approach since cleavage of the ligand from the intermediate molecule is problematic.

The formation of the new compounds can be accomplished by any concept for synthesis known in the art. However, methods of combinatorial chemistry, in particular solid phase combinatorial chemistry, are preferred.

The replacing fragment of step (e) may be a ligand itself. For the selection of such a ligand used as a replacing fragment, the same criteria apply as for the selection of the ligands used in step (a). Alternatively, the replacing fragment may be a combination of a ligand and an intermediate molecule (the latter being equal to or different from the ones used in step (a). Thus, for example, the ligands selected in step (d) may be combined with a ligand-anchor-conjugate carrying a different ligand and immobilized together on the solid support for another screening step. In another embodiment, the ligands of step (d) may be combined with a ligand to which a ligand-tag is bound, and this combination is immobilized via suitable anchor structures on the support as described above. Of course, the same result may be achieved if a ligand is used as a replacing fragment which comprises a functional group allowing it to form a covalent bond with an intermediate molecule as defined above. In any of the above cases, a new ligand structure with increased molecular weight, usually increased complexity, and potentially increased affinity, is provided by the replacement.

The replacing fragment of step (e) may be identical for each of the ligands selected in step (d). However, it is preferred to use replacing fragments varying in their structure, so that a highly diverse group of compounds is provided in order to facilitate the identification of highly active structures.

It must be re-emphasised in this context that it is an important feature of the present invention to covalently couple the replacing fragment to the original ligand at the same position where the ligand was attached to the common intermediate molecule in order to retain the ligand's orientation. Since the member molecules of the binding matrix should only vary in their ligand structures, the bond at which they need to be modified in step (e) is known. In case a plurality of ligands, e.g. resulting from a unitary synthesis protocol, shares one functional group at which they are bound with the intermediate molecule (e.g. a carboxylic functional group) it is usually advantageous to consider such a group as being contained in the ligand structure, and not as a part of the common intermediate molecule.

If the replacing fragment comprises an intermediate molecule suitable for the immobilisation of the new compound obtained in step (e), steps (a) to (e) may be repeated. This strategy allows the improvement of the ligand structure in terms of its affinity to the target while continuously extending the structure of the ligand.

However, in order to render the obtained results comparable and to verify that the final ligands in their free state give rise to similar results as in their immobilized state, the method according to the invention usually comprises as a last step (f) the determination of the affinity of the ligands. This final step may be carried out directly after the first replacement of the common intermediate molecule if the replacing fragment does not provide a structural element intended to immobilize the new ligand on a screening support. Usually, affinity determination is carried out if step (e) yields a ligand-ligand combination which can be expected to have sufficient affinity towards the given target. For the affinity determination, the combination of the ligand and the replacing fragment/ligand should be present in solution or as a dispersion.

In this step, an absolute value for the affinity of the ligand—target complex, such as its dissociation constant K_{D}, its association constant K_{A} or the inhibitory constant of the ligand Kₗ or its IC₅₀ value, is determined in solution with the ligand in a free, non-immobilized form. Such values, obtained according to conventional methods e.g. from the equilibrium in solution between free ligands and targets on the one hand and ligand—target complexes on the other hand are characteristic indicators for the in vivo effectiveness of a chosen ligand. Competition assays are frequently used.

For better comparison of the results of the ligand—target affinity tests in solution from ligands before and after introduction of their replacing fragment, the functionality for immobilisation of the ligand to be tested without an replacing fragment can be slightly modified. Such a modification prevents the functional group of the original ligand, which is blocked if the ligand is in an immobilised state and which is used to bind with the replacing fragment to contribute significantly to the determined affinity if the ligand is tested in solution. As a consequence, the influence of this functional group, which is no longer available for ligand target interaction in a ligand bound to a replacing fragment on the comparison of the affinities can be minimised. Preferably, functionalities which may otherwise contribute to electrostatic interactions between ligand and target like amino- or carboxylic groups are modified to reduce their polarity. For example, in case of testing the ligands in assays under pH-neutral conditions, the comparability between ligands before and after introduction of their replacing fragment can be improved when a carboxylic function, which would act as carboxylate anion under the assay conditions, is converted to a neutral acting carboxamide group.

### EXAMPLE

Solid Phase Screening

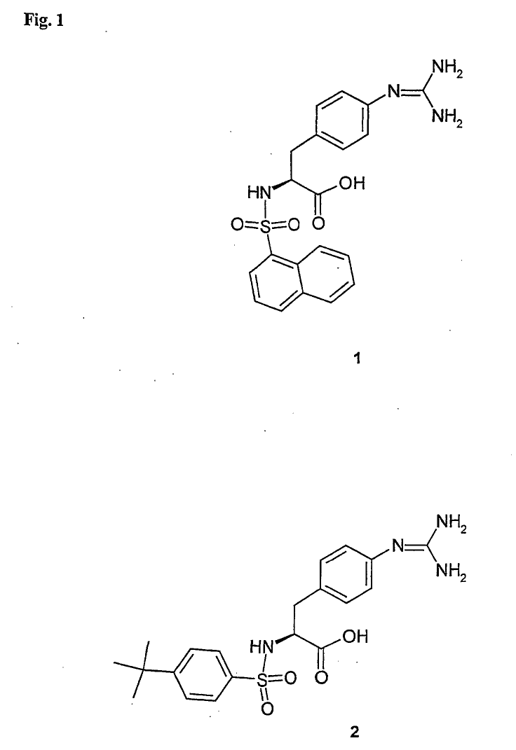

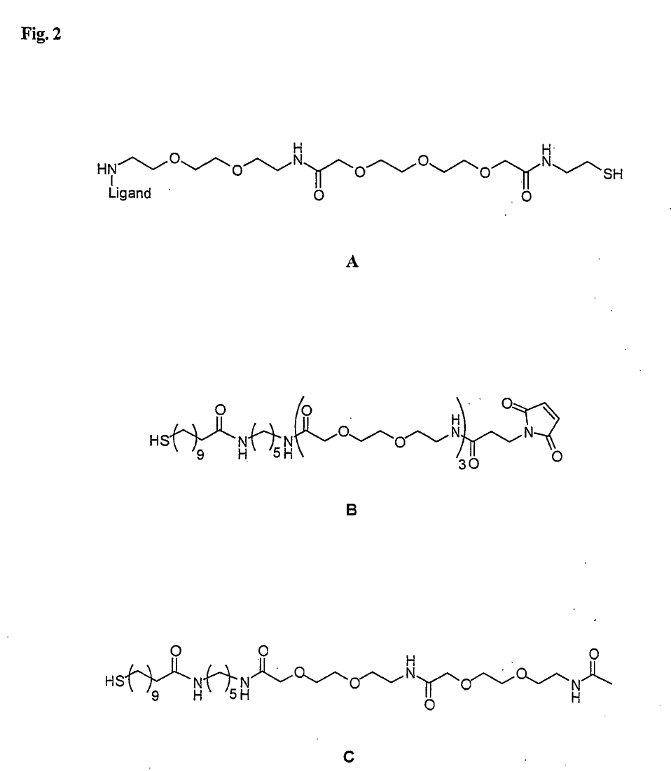

Starting compounds 1 and 2 (FIG. 1) were covalently bound to the amino function of a ligand tag via the carboxylic acid function. The product corresponds to general formula A in FIG. 2. The synthesis was carried out on solid phase. Using the Fmoc protecting group technique, the ligand tag was immobilised in advance by means of trityl linkers, then the amino acid was coupled to the solid phase. Afterwards the corresponding sulfonic acid was reacted in the form of its chloride and the product cleaved by means of TFA.

A gold chip (5×5 cm) was incubated with a 1:25 mixture of maleimide-thiol anchor molecule B (FIG. 2) and a diluting component C (FIG. 2) in ethylene glycol and 1% TFA (total concentration 1.0 mM). The anchor molecule and the diluting component were synthesized as described in DE 100 27 397.1 (Example 1 and 2). The chip was then washed several times in methanol/1% TFA and subsequently in H₂O (pH 7.0) and dried under a nitrogen flow.

The ligandtag conjugates (ligands carrying a ligand tag) were placed on the chip which had been treated in advance in this way by means of a pinning tool. The ligand tag conjugates which are placed on the surface are dissolved in a 40 μM solution of 0.2 M phosphate buffer (Pi), 5 mM EDTA and 10% (v/v) EG (ethylene glycol), pH 7.0. The pinning tool deposits about 5 nl per spot so that a high excess of the ligand tag conjugate as compared with the surface-bound maleimide group is guaranteed in each spot and, thus, complete coverage of the maleimide groups can be achieved. The maleimide groups in the uncovered areas were subsequently saturated by incubating the chip in 0.2 M Pi, pH 7.0, 10 mM mesh (beta-mercaptoethanol) for 30 min.

This chip was then incubated overnight in BSA (bovine serum albumine) blocking solution (50 mM Tris/HCl, 150 mM NaCl, 5 g/l BSA, 0.05% (v/v) Tween-20, pH 7.3). Potential binding partners of the target molecule thrombin were analysed in the subsequent immunoassay: for this purpose, the chip was first incubated for 4 hours in 10 nM thrombin in blocking solution. After washing it twice for 2 minutes in blocking solution, a 1:1000 dilution of a polyclonal anti-thrombin antibody was incubated with the chip for 2 hours. After washing it again twice in blocking solution, an anti-rabbit antibody POD conjugate was incubated with the chip for 2 hours in order to detect binding reactions. Finally the chip was washed twice in TBST for 2 minutes. The chemiluminescence reaction was detected by reacting the Lumi-Light-Plus substrate in the Lumi Imager (Roche). Light-colored spots indicate the binding of thrombin. In parallel with this reaction, a second chip was treated in an identical way, except that it was not incubated with thrombin. This chip serves as a negative control in order to differentiate binding reactions which have not been created by thrombin, but by primary or secondary antibodies. The negative control did not show any signals beyond noise. Since each compound has a specific spatial coordinate on the chip, the spots can be assigned to the respective chemical structures.

Optimisation of Ligands by “Tag Replacement” and Determination of their Inhibitory Constants

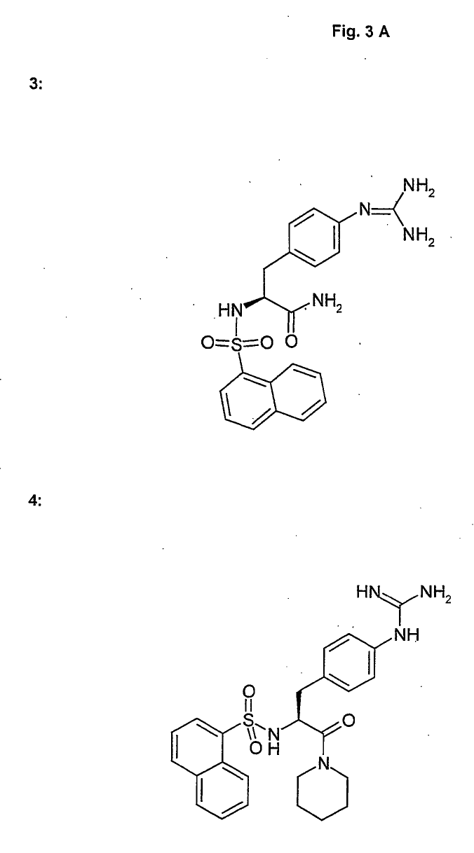

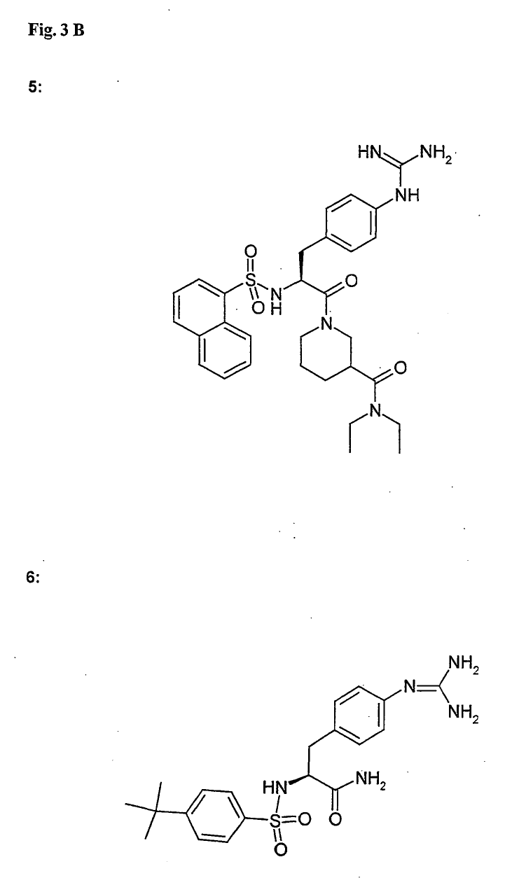

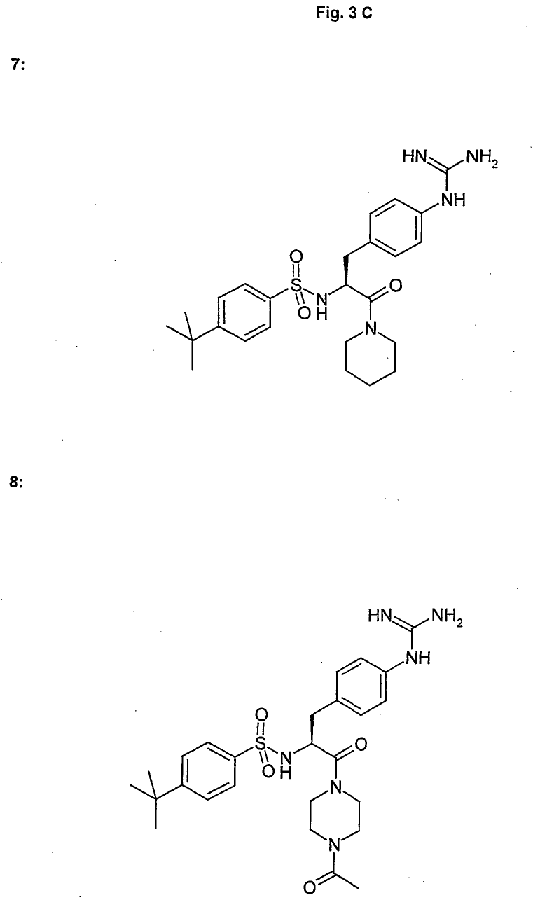

As the starting compounds, the binary thrombin ligands identified in solid phase screening, the tags of which had been replaced by various amines, were used. The corresponding compounds were synthesised as follows (FIG. 3). For better comparison, not starting compounds 1 and 2, but the carboxamide analogon thereof was used in the subsequent comparatory competition assay. Compound 3 corresponds to 1 and 6 corresponds to 2.

Compounds 6 and 3 were prepared by means of solid phase synthesis.

Fmoc-L-bis(tert-butyloxycarbonyl)guanidinophenyli alanine was coupled to NovaSyn TGR resin (Novabiochem) using standard protocols of peptide synthesis via activation with N,N′-diisopropyl carbodiimide and N-hydroxy benzotriazole. After cleaving the Fmoc protective group with 20% piperidine in dimethyl formamide (DMF), the resin was divided into two portions.

For synthesising 6 and 3, respectively, the resin was shaken for 30 minutes with 5 eq. 1-naphthylsulfonyl chloride and 4-tert-butylphenylsulfonyl chloride, respectively, and 5 eq. each of triethyl amine in DMF (c=0.1 M).

The cleavage from the resin was effected by means of 95% trifluoroacetic acid with 5% triethyl silane.

Both compounds were purified by means of reverse phase HPLC and analysed by means of analytical LC/MS.

Compounds 4, 5, 7 and 8 were prepared according to Bradley et al. (1996) J. Am. Chem. Soc., 118, 3055-3056:

Fmoc-L-bis(tert-butyloxy carbonyl)guanidinophenyl alanine was coupled to the described resin and after Fmoc cleavage 1-naphthylsulfonyl chloride and 4-tert-butylphenyl sulfonyl chloride were bound thereto. After activating the respective resin portions with iodoacetonitrile, compounds 4, 5, 7 and 8 were obtained by incubating the resin portions with piperidine and N,N-diethyl nipecotamide, respectively. The compounds were purified by means of reverse phase HPLC and analysed by means of analytical LC/MS.

The inhibitory properties of these substances were then analysed in a thrombin competition assay (determination of the inhibitor constant Kᵢ).

| Starting compound/Kᵢ | | |
|---|---|---|
| (M) | Compound 1/Kᵢ (M) | Compound 2/Kᵢ (M) |
| 3/6 · 10⁻⁵ | 4/1 · 10⁻⁶ | 5/6 · 10⁻⁶ |
| 6/6 · 10⁻⁶ | 7/6 · 10⁻⁷ | 8/2 · 10⁻⁶ |

The thrombin activity was determined at 20° C. and pH 7.4 using the fluorogenic substrate Tos-GPR-AMC (Bachem, 11365, λexc. =370 nm, λem. =450 nm). The reaction mixture contains 20 μM substrate, 0.1-100 μM inhibitor and 100 μM human thrombin in a total volume of 200 μl HBS (10 mM Hepes, 150 mM NaCl, 0.005% Tween 20). After 5 minutes of incubating the enzyme in advance with the inhibitor, the reaction is started by adding substrate and the fluorescence intensity is measured for 10 minutes in intervals of 1 minute. With competitive inhibition, the K value is calculated as follows:

v₀/vᵢ=1+/Kᵢ

v₀=initial speed of the reaction

vᵢ=initial speed of the reaction in the presence of the inhibitor

l=inhibitor concentration

The Kᵢ values of the compounds produced by tag replacement show increased affinity (lower Kᵢ value) as compared with thrombin.

1. A method for the identification of active compounds interacting with a target molecule, comprising the steps of:
a) forming a binding matrix comprising at least two different ligands on a solid support by immobilising said ligands via a common intermediate molecule on the support;
b) contacting a target of interest with said binding matrix;
c) parallely determining a binding value of the ligand/target interaction for each type of ligand comprised in the binding matrix;
d) selecting those ligands the binding value of which in an immobilised state with the target exceeds a predetermined threshold;
e) replacing the common intermediate molecule by a replacing fragment in order to form new ligands of increased molecular weight, with the replacing fragment being coupled to the ligand(s) of step (d) at the common binding position between the ligand and the intermediate molecule; and
f) determining the affinity of the ligands formed in step (e) towards the target.

2. The method of claim 1, wherein steps (a) to (e) are repeated once or several times with the new ligands of step (e).

3. The method of claim 2, wherein the replacing fragment used in step (e) comprises a structure which optionally serves as an intermediate molecule to immobilise the ligands on the support in a repeated step (a).

4. The method according to any of claims 1 to 3 wherein a plurality of different replacing fragments, optionally comprising a common intermediate molecule, are used in step (d).

5. The method according to claim 1, wherein at least 1000, preferably at least 9000 different types of ligands are used for the formation of the binding matrix.

6. The method according to claim 1, wherein one or more of: the ligands of step (a), the replacing fragments of step (d), and the new ligands of step (d) obey the Lipinsky rule.

7. The method according to claim 1, wherein the binding matrix is formed as an array of discrete fields, each carrying one type of ligand.

8. The method according to claim 1, wherein the ligands of step (a) are immobilised via intermediate molecules capable of forming a self assembling monolayer.

9. The method according claim 8, wherein anchor structures capable of forming a self assembling monolayer are immobilised on the solid support and subsequently covalently bound to the ligands to provide the binding matrix.

10. The method according to claim 9, wherein the ligands are bound to the anchor structures via ligand tags so that the anchor structures and the ligand tags form the intermediate molecules.

11. The method according to claim 8, wherein the monolayer comprises dilution components in order to separate the ligands in the binding matrix.

12. The method according to claim 11, wherein: a) the surface of the support comprises a gold layer; and b) the intermediate molecules and the dilution components comprise a thiol functional group to allow their attachment to the support.

13. The method according to claim 1, wherein the affinity determination of step (f) is carried out in solution.

14. The method according to claim 13, wherein the affinity determination of step (f) is carried out as a competition-assay.

15. The method according to claim 1, wherein the target molecule is a protein.
